(19) United States
(12) Patent Application Publication
(10) Pub. No.: US 2006/0223750 A1
(43) Pub. Date: Oct. 5, 2006

(54) Agents and methods for enhancing photodynamic therapy

(75) Inventors: James A. Burke, Santa Ana, CA (US); Elizabeth T. Syage, Huntington Beach, CA (US); Michael E. Garst, Newport Beach, CA (US); Larry A. Wheeler, Irvine, CA (US)

Correspondence Address: ALLERGAN, INC., LEGAL DEPARTMENT, 2525 DUPONT DRIVE, T2-7H, IRVINE, CA, 92612-1599, US

(73) Assignee: Allergan, Inc., Irvine, CA (US)

(21) Appl. No.: 11/096,886

(22) Filed: Apr. 1, 2005

(51) Int. Cl. A61K 38/17; A61K 38/05; A61K 31/739; A61K 31/409; A61K 31/555; C07K 14/74; C07K 5/06

(52) U.S. Cl. 514/12; 514/54; 514/185; 514/410; 514/18; 530/350; 536/53; 530/331

(57) ABSTRACT

A chemical conjugate for administration to a patient undergoing photodynamic therapy includes a photoactive compound coupled to a leakage reducing agent that is structured to reduce leakage of the photoactive compound from a patient's vasculature. The leakage reducing agent may be a bulking agent to reduce, for example, sterically reduce, the permeability of the photoactive compound through the blood vessel, or it may be a ligand that binds to endothelial cells of a patient's choriocapillaries. The conjugate may be used in methods of reducing secondary damage associated with photodynamic therapy.

### BACKGROUND

Choroidal neovascularization (CNV) involves abnormal growth of blood vessels from the choroid through Bruch's membrane to the region beneath the retina. The abnormal blood growth results in leakage and bleeding into the subretinal space, which may result in scar formation beneath the macula of the retina and a loss of vision. Choroidal neovascularization may be associated with macular degeneration, such as age related macular degeneration, and disorders of the eye, including ocular histoplasmosis syndrome, myopia, diabetic retinopathy, and inflammatory diseases, among other disorders, diseases and conditions.

Traditionally, CNV has been treated by occluding the abnormal blood vessels with thermal energy transmitted from a laser. Thermal photocoagulation of the blood vessels undesirably results in full-thickness retinal damage, as well as damage to medium and large choroidal blood vessels. More recently, lasers have been used to provide more selective closure or occlusion of the abnormal blood vessels. One example includes the use of photosensitive or photoactive chemical compounds that are activated by electromagnetic energy transmitted from a laser; this treatment is commonly referred to as photodynamic therapy. With photodynamic therapy, a patient typically receives an injection of a photoactive compound. The photoactive compound accumulates within the CNV at which point a laser is used to direct relatively low power electromagnetic energy of a specified wavelength particular for the photoactive compound. Using a low power laser reduces the potential of thermal damage associated with traditional techniques. When the photoactive compound is activated by absorbing the energy from the laser, reactive ion species, such as free radicals, are generated which cause cellular destruction and result in occlusion of the CNV.

Although the photodynamic therapy provides improved results compared to thermal photocoagulation, clinical evidence exists indicating that photodynamic therapy is associated with a transient leakage of fluid from the choriocapillaries into the choriocapillaris, and a transient decrease in neurosensory function.

Thus, there remains a need for additional agents and methods for photodynamic therapy that enhance the efficacy of the laser treatment and reduce secondary damage associated with photodynamic therapy.

### SUMMARY

Conjugates of a photoactive compound and a leakage reducing agent coupled to the photoactive compound are disclosed. The conjugates are effective to enhance photodynamic therapy by reducing permeability, for example, leakage, of the photoactive compound from blood vessels. For example, the present conjugates are advantageously effective to reduce permeability of the photoactive compound from a blood vessel of a patient relative to permeability of the photoactive compound from a blood vessel of a patient after the photoactive compound without the leakage reducing agent is administered to the patient. Using the conjugates disclosed herein, secondary damage associated with conventional photodynamic therapy is reduced. The conjugates may be administered to a patient in a pharmaceutical composition which permits systemic delivery of the conjugate to a target site for photodynamic therapy. The conjugates find particular use in photodynamic therapy of choroidal neovascularization associated with ocular disorders, diseases, conditions and the like.

The photoactive compounds of the conjugates are chemical compounds that absorb a relatively narrow band of electromagnetic energy transmitted from a laser. Upon irradiation, the photoactive compound becomes activated to generate reactive molecules that destroy cellular structures in the vicinity of the photoactive compound. Activation of the photoactive compounds of the conjugates thus results in the occlusion of targeted vascular sites.

The leakage reducing agents of the conjugates may either be bulking agents that do not have a specific affinity for a particular cell types, or may be a ligand for endothelial cells and/or other chemical entity that permits the conjugates to be attached to the endothelial cells without leaking out of blood vessels. For example, the conjugate may comprise a photoactive compound coupled to a bulking agent that is sized to reduce, for example, sterically reduce, permeability of the photoactive compound through a blood vessel to reduce the amount of the photoactive compound outside of the blood vessel relative to administering the photoactive compound without the bulking agent to the patient.

The photoactive compounds and the leakage reducing agents may be coupled together, for example, and without limitation, may be covalently bonded together, or may include a linker between the photoactive compound and the leakage reducing agent. The linker may provide the ability to augment the therapeutic effects caused by absorption of electromagnetic energy by the photoactive compound of the conjugates.

The conjugates may be administered to a patient to reduce secondary damage associated with photodynamic therapy by reducing leakage of the photoactive compound from the vasculature of the patient. In addition, methods disclosed herein may include one or more additional steps of administering other agents to reduce secondary damage associated with photodynamic therapy. For example, one or more neuroprotective agents may be administered separately or in conjunction with administration of the conjugate to the patient. Or, one or more additional photoactive compounds that are smaller than the conjugates may be administered to the patient after the administration of the conjugates to provide an increased local concentration of photoactive compounds within a target site of a patient.

Any feature or combination of features described herein are included within the scope of the present invention provided that the features included in any such combination are not mutually inconsistent as will be apparent from the context, this specification, and the knowledge of one of ordinary skill in the art.

Additional advantages and aspects of the present invention are apparent in the following detailed description and claims.

### DETAILED DESCRIPTION

The present invention is directed to agents and methods for photodynamic therapy, such as photodynamic therapy used to treat choroidal neovascularization (CNV). More particularly, the agents and methods disclosed herein attempt to reduce secondary damage associated with photodynamic therapy of CNV. Agents and methods are disclosed that attempt to reduce leakage of photoactive compounds from blood vessels to provide more localized distribution of photoactive compounds in the blood vessels, thereby providing a more localized target for the photodynamic therapy. The agents and methods may enhance the efficacy and safety of photodynamic therapy.

Agents for reducing secondary damage associated with photodynamic therapy generally comprise a conjugate of a photoactive compound and a leakage reducing agent. The leakage reducing agent is coupled to the photoactive compound and is stuctured to reduce leakage of the photoactive compound from a blood vessel of a patient (human or non-human animal) to which the conjugate has been administered. In the case of treatment of CNV, the leakage reducing agent is structured to reduce leakage of the photoactive compound from one or more choriocapillaries into the choriocapillaris of an eye of a patient. Thus, in contrast to existing agents and methods, the present conjugated photoactive compounds exhibit reduced leakage, or even barely, if at all, leak, that is exhibit substantially reduced or even substantially no leakage, through the blood vessel into the interstitial space. Because the photoactive compound of the conjugates disclosed herein is more localized within the blood vessels, electromagnetic energy transmitted from a laser is primarily absorbed by the photoactive compound within the patient's vasculature, as opposed to photoactive compound that has passed out of the blood vessels into the interstitial space. Furthermore, because the conjugates have a tendency to accumulate within the CNV due to, among other things, differences in blood flow rate through the choriocapillaries, the conjugates disclosed herein provide one or more improvements to the efficacy of treatment provided by photodynamic therapy relative to the use of the photoactive compound without the leakage reducing agent.

The leakage reducing agent of the conjugate may comprise a bulking agent or it may comprise a ligand that binds to endothelial cells of choriocapillaries, or the leakage reducing agent may comprise a combination of one or more bulking agents and one or more endothelial cell ligands. A bulking agent, as used herein, is a molecule or moiety that is relatively large, and is sized to reduce, and preferably prevent, the photoactive compound to which the bulking agent is coupled from passing through fenestrations of blood vessels, and more particularly fenestrations of choriocapillaries of an eye of the patient. The bulking agent of the conjugate may comprise a lipid, a fatty acid, and/or a carbohydrate, or combinations thereof.

In one embodiment, the bulking agent comprises a polysaccharide (e.g., a chain of two or more sugars), such as a dextran. Carbohydrate bulking agents may have a molecular weight between about 60 and about 120 kilodaltons, and preferably between about 70 and about 100 kilodaltons. In one embodiment, the bulking agent of the conjugate has a molecular weight of about 80 kilodaltons. These sizes of the carbohydrate bulking agents appear to provide the desired physical effects to reduce the photoactive compound from leaking out of the blood vessels.

When the bulking agent used in the conjugate is a lipid or a fatty acid, it may be desirable to couple a sufficient number of lipid or fatty acid molecules to the photoactive compound to obtain the desired size of the conjugate to reduce leakage of the photoactive compound from the choriocapillaries. For example, multiple lipids or fatty acids may be coupled at different sites to the photoactive compound to create a conjugate that is physically too large to pass through blood vessel fenestrations. Alternatively, or in addition, lipids and fatty acids may be employed by coupling the lipids or fatty acids to other lipids and fatty acids. The bulking agent achieves an effect of reducing leakage through blood vessel fenestrations by sterically hindering the ability of the conjugate from passing through the fenestrations.

Accordingly, conjugates having a bulking agent as a leakage reducing agent do not have a significant affinity for a specific cellular target, or in other words, the conjugates are non-selective for cellular targets. Typical fatty acids may have between about twelve and about twenty-four carbons, but other fatty acids may have longer carbon chains. Some examples of fatty acids that are conjugated to a photoactive compound include, and are not limited to, palmitic acid, steric acid, oleic acid, linoleic acid, and linolenic acid. Examples of lipids that may be used in the conjugates disclosed herein include, and are not limited to, triglycerides, phospholipids, and sterols.

A ligand that binds to endothelial cells preferably is a ligand that specifically binds to endothelial cells, or preferentially binds to endothelial cells as compared to non-endothelial cells. The ligand may be a natural or synthetic molecule or moiety, and may be a small chemical compound, a protein, or a nucleic acid. Examples of ligands include, without limitation, antibodies or antibody fragments of one or more markers (e.g., protein or carbohydrate markers) present on the cell surface of endothelial cells, peptides having one or more tripeptide amino acid sequences of RGD (RGD peptides), RGD peptide analogs, integrins or integrin receptors, such as receptors that bind to the α_{V}β₃ integrins that are expressed on endothelial cells, and/or carbohydrates that bind to endothelial cells, such as the blood group antigen Sialyl Lewis X (SLeX), and the like and mixtures thereof.

The photoactive compound of the conjugate is a chemical compound having a structure that allows the compound to absorb energy, such as electromagnetic energy, that is transmitted from a laser. When the photoactive compound absorbs the energy from the laser, one or more reactive ion species, such as free radicals, are formed that cause cellular damage and result in the destruction of the CNV. Examples of photoactive compounds include, and are not limited to, porphyrins, hematoporphyrins, hematoporphyrin derivatives, pheophorbides, derivatives of pheophorbides, benzoporphyrins, benzoporphyrin derivatives, such as verteporfin, bacteriochlorins, purpurins, merocyanines, porphycenes, tricarbocyanines, such as indocyanine green, and combinations thereof and mixtures thereof.

These, as well as other photoactive compounds, are described in U.S. Pat. Nos. 5,028,621; 4,866,168; 4,935,498; 4,649,151; 5,438,071; 5,198,460; 5,002,962; 5,093,349; 5,171,741; 5,173,504; 4,968,715; 5,190,966; 5,314,905; 5,587,371; 5,798,349; 5,587,479; 6,225,303; U.S. Publication No. 2002/0094998, and PCT Publication No. WO 01/58240, all of which are hereby incorporated in their entireties by reference herein. Preferably photoactive compounds are compounds that may be administered to a patient without causing substantial or significant undesirable side effects, and that absorb certain wavelengths of energy transmitted from an electromagnetic energy source, such as laser, that do not cause substantially or significant undesirable thermal damage. In other words, the effects provided by the laser treatment are due primarily to the generation of reactive molecules from the photoactive compound by absorption of energy from the laser.

The photoactive compound and the leakage reducing agent may be covalently coupled together using conventional techniques that are well known to persons of ordinary skill in the art. For example, a covalent bond may be created between a photoactive compound and a bulking agent by using a dehydrating agent, such as carbodiimide (e.g., 1-ethyl-3-(3-dimethylaminopropyl)carbodiimide (EDAC)) and conventional techniques.

Alternatively, the conjugate may comprise a linker located between the photoactive compound and the leakage reducing agent. For example, an affinity linker could be coupled to the leakage reducing agent, such as a bulking agent, such as a dextran, and the free end of the linker could react with the photoactive compound to make a covalent attachment. In one embodiment, the free end of the linker comprises a malimide or an imidate. The linker may be provided so that the linker is non-covalently attached to the leakage reducing agent, and covalently bonded to the photoactive compound, or the linker can be non-covalently attached to the photoactive compound and covalently attached the leakage reducing agent, or the linker may be covalently bonded to both the photoactive compound and the leakage reducing agent.

The linker may be structured to augment distribution of the photoactive compound within the vasculature of the patient. For example, the linker may act as a tether between the photoactive compound and the leakage reducing agent, and the length of the tether can be varied or adjusted to affect properties, such as distribution and/or quenching of the photoactive compound.

When coupling the leakage reducing agent to the photoactive compound, it is desirable that the energy absorbing properties of the photoactive compound are not significantly augmented or altered so that the photoactivity of the conjugate is similar, if not identical, to the photoactivity of the photoactive compound without the leakage reducing agent.

Dextran-porphyrin conjugates may be prepared by any suitable technique known to those of ordinary skill in the art, for example, by using a spacer such as a glycyl spacer, an aminobutyric acid spacer or an aminocaproic acid spacer for attaching the porphyrin to the dextran.

Techniques for preparing dextran conjugates are generally described in the following publications: Konieczny, M., et al, Search for Carriers for Non-covalent Binding of Interferon among 1,3,5-Triazine Derivatives of Dextran, Archivum Immunologiae et Therapiae Experimentalis (1982) 30; Remon, J. P. et al, Polymer-Drug Combinations: Pinocytic Uptake of Modified Polysaccharides containing Procainamide Moieties by Rat Visceral Yolk Sacs Cultured In Vitro, J. of Controlled release, 1 (1984) 47-56; and E. Schacht, et al., Polymer-drug Combinations: Synthesis and Characterization of Modified Polysaccharides Containing Procainamide Moieties, J. Controlled Release, 1 (1984) 33-46. The entire disclosure of each of these publications being incorporated herein in its entirety by this specific reference thereto.

By way of example, and not limitation, a dextran-vertoporfin conjugate may be made by dissolving 0.65 g (0.004 mole) of dextran (obtained from Amersham International, U.K.) in 20 ml of water to which 1 ml of aqueous vertoporfin solution is added (mole ratio of dextran to vertoporfin is 20:1) Afterwards, 0.25 ml of 40% NaOH and 3 g NaCl is added and the mixture is stirred at room temperature for 5 hours. The reaction mixture is purified by dialysis. The content of the dialyzation sack is condensed through a membrane and evaporated to dryness on a vacuum evaporator. The resulting polymeric-drug conjugate is useful in accordance with the methods of the present invention. Other methods of preparing suitable conjugates are readily known by persons of ordinary skill in the art.

The conjugates disclosed herein are preferably provided in a pharmaceutical composition or formulation for administration to a patient. The particular nature of the composition will depend on, among other things, the mode of administration to the patient, and the nature of the conjugate being administered, as understood by persons skilled in the art. The compositions may be provided with any pharmaceutically accepted excipient, such as water, saline, dextrose, glycerol, and the like and mixtures thereof.

The compositions may be administered systemically or topically. For example, the compositions may be administered by intravenous, subcutaneous, intramuscular, and/or intraperitoneal injection, or by topically applying the composition to the patient's skin for transdermal delivery into the patient's vasculature, or by administering the composition to the patient's eye, or orally. Compositions intended for topical administration may include a permeability enhancing agent that facilitates the delivery of the conjugate into the circulatory system. Examples of suitable permeability enhancing agents include dimethylsulphoxide (DMSO) and/or liposomes, and the like, among others, and mixtures thereof. For example, in one embodiment, a permeability enhancing agent is administered to a patient to enhance the topical administration of the conjugate to the patient relative to a similar method without the step of administering a permeability enhancing agent to the patient.

In addition, one or more neuroprotectants may be administered to the patient in conjunction with the photodynamic therapy. Neuroprotectants may be administered in a separate formulation or in the same formulation containing the conjugate. Neuroprotective agents preferably preserve the cellular, biochemical, and physiological properties of the neurons. Examples of neuroprotective agents include, without limitation, anti-excitotoxic agents, such as glutamate receptor (e.g., NMDA receptor) modulators (such as, MK-801, N4K-801, memantine), calcium channel blockers, and inhibitory receptor modulators (such as GABA receptor agonists, including, but not limited to, anesthetics, such as barbiturates; benzodiazepines, such as zolpidem; and alcohol, such as ethanol). Anti-excitotoxic agents preferably reduce or prevent excessive increases in intracellular calcium concentration.

Neuroprotective agents also include adenosine receptor modulators, adrenergic receptor modulators (such as, α2-receptor agonists, brimonidine, beta-blockers, etc.), glutamate uptake modulators, dopamine receptor modulators, ion channel modulators (such as, sodium or hydrogen), downstream intracellular signal modulators (such as, COP-1), prostaglandins (such as EP2 agonists), ryanodine receptor agonists (calcium release from intracellular stores), cytokines, neurotrophic and/or nerve growth factors, such as nerve growth factor (NGF) including NGFα, brain derived neurotrophic factor (BDNF), ciliary neurotrophic factor (CNTF), bone-derived growth factor (BDGF), neurotrophin-3 (NT-3), neurotrophin-4/5 (NT-4/5), pigment epithelium derived factor, vitamin C, steroids, non-steroidals, cyclosporins, drugs that are active in ischemia/reperfusion assays, monoamine oxidase inhibitors (MAOIs), carbonic anhydrase inhibitors (such as acetazolamide), pump inhibitors (such as, amiloride), free-radical scavengers, nitric oxide synthetase inhibitors, and hormones.

The dosage of the photoactive compound that is administered to a patient may vary, according to the activity of the specific compound(s) chosen, the formulation, and whether the compound is joined to a carrier and thus targeted to a specific tissue as described above. When using green porphyrins, dosages are usually in the range of about 0.1-50 mg/M² of body surface area; more preferably about 1-10 mg/M² or about 2-8 mg/M². Parameters to be considered when determining the dosage include the duration and wavelength of the light irradiation, the nature of the photochemical reaction induced by the light irradiation, and the dye-to-laser time period.

Electromagnetic energy is directed to target sites for a sufficient time after the administration of the conjugate of the photoactive compound and leakage reducing agent so as to permit the conjugate to reach its target tissue. Upon being irradiated with the wavelength(s) appropriate to the compound(s) chosen, the compound enters an excited state and is thought to interact with other compounds to form highly reactive intermediates which can then destroy the target endothelial tissue, causing platelet aggregation and thrombosis. Fluence of the irradiation may vary depending on factors such as the depth of tissue to be treated and the tissue type—generally it is between about 25 and about 200 Joules/cm². Irradiance typically is between about 150 and about 900 mW/cm², but can also vary somewhat from this range.

Light treatment may be given as soon as about 5 minutes following administration of the conjugate; however, light treatment may be given at about 2 hours to about 6 hours after administration of the conjugate. Among other things, due to the reduced or decreased leakage associated with the conjugates disclosed herein, it has been discovered that the conjugates remain in the CNV for extended periods of time as compared to photoactive compounds without leakage reducing agents. In a preferred embodiment, the photoactive drug is administered intravenously.

By administering the conjugates disclosed herein to a patient undergoing photodynamic therapy, secondary damage associated with leakage of photoactive compounds from choriocapillaries is reduced. Among other things, the photoactive compound appears to stay more confined to the CNV and does not appear to significantly permeate through fenestrations of choriocapillaries. The therapeutic effects provided by the electromagnetic energy absorption by the photoactive compound of the conjugate are improved because the patients receiving the photodynamic therapy do not exhibit a noticeable reduction in neurosensory function or persistent leakage from choriocapillaries. Advantageously, the conjugate is structured so that a major portion of the electromagnetic energy emitted from a laser is absorbed by the photoactive compound of the conjugate located in the CNV.

The methods herein disclosed may also comprise a step of administering a second photoactive compound that has a smaller size (e.g., smaller molecular weight) than the conjugate administered to the patient. Administering a combination of two or more photoactive compounds of varying sizes appears to provide greater localization of the photoactive compound within blood vessels of a target site thereby creating a more effective treatment of the CNV. While not wishing to be bound by any particular theory or mechanism of action, it appears that administering one or more conjugates, as disclosed herein, followed by administration of one or more smaller photoactive compounds, permits the conjugates to sufficiently occlude blood vessel fenestrations for an amount of time that allows the smaller photoactive compounds to reach the target site and not permeate through the fenestrations. The combination of the photoactive compounds appears to provide more effective (e.g., more focused) treatment by the laser. In certain embodiments, it is desirable to use a conjugate and a smaller photoactive compound that have similar energy absorbing properties (e.g., each absorbs similar wavelengths of energy).

The following non-limiting examples illustrate certain aspects of the present invention.

### EXAMPLE 1

A 74 year old patient presents with “wet” age-related macular degeneration (ARMD) in the foveal region of the right eye, and his condition is found to be suitable for photodynamic therapy (PDT). The day of scheduled PDT treatment, the patient is administered a composition of 6 mg/M2 of verteporfin coupled to dextran-80. Thirty minutes after the start of the infusion, the patient is administered Irradiance of 600 mW/cm2 and total fluence of 75 Joules/cm2 from an Argon light laser. The treatment requires irradiation of the optic nerve.

Evaluation of neural health is assayed 1 week, 4 weeks, and 12 weeks following treatment by visual inspection of the retina and test of visual acuity. The affected areas of the retina appear healthy with no whitening (indicating lack of discernable retina damage) one week following PDT treatment. This trend continues throughout the monitoring period. Fluorescein angiography at same time points shows minimal leakage in the treated tissue after one week, and this minimal leakage continues throughout the monitoring period. No evidence of renewed neovascularization can be seen 12 weeks following PDT treatment. Additionally, no evidence of optic nerve axon loss can been seen. Tests of visual acuity 4 and 12 weeks following PDT with the conjugate treatment show no discernable loss of vision as a result of the treatment.

### EXAMPLE 2

A patient with similar symptoms as the patient in Example 1 is treated with a similar protocol as the patient Example 1, except the patient is administered a composition containing a conjugate which comprises 6 mg/M2 of verteporfin coupled to linoelic acid in a 10:1 molar ratio. Similar results were obtained.

Various publications and/or references have been cited herein, the contents of which, in their entireties, are incorporated herein by reference.

While this invention has been described with respect to various specific examples and embodiments, it is to be understood that the invention is not limited thereto and that it can be variously practiced with the scope of the following claims.

What is claimed is:

1. A conjugate for reducing secondary damage associated with laser treatment of choroidal neovascularization, the conjugate comprising a photoactive compound coupled to a bulking agent so that the size of the conjugate is greater than the size of the photoactive compound without the bulking agent, and the size is sufficient to reduce permeability of the photoactive compound from a blood vessel of a patient after the conjugate is administered to the patient relative to the permeability of the photoactive compound from a blood vessel of a patent after the photoactive compound without the bulking agent is administered to the patient.

2. The conjugate of claim 1, wherein the photoactive compound comprises a porphyrin.

3. The conjugate of claim 1, wherein the photoactive compound comprises verteporfin.

4. The conjugate of claim 1, wherein the photoactive compound is selected from a group consisting of hematoporphyrins, hematoporphyrin derivatives, pheophorbides, derivatives of pheophorbides, bacteriochlorins, purpurins, merocyanines, porphycenes, and combinations thereof.

5. The conjugate of claim 1, wherein the bulking agent comprises a lipid.

6. The conjugate of claim 1, wherein the bulking agent comprises a fatty acid.

7. The conjugate of claim 1, wherein the bulking agent comprises a carbohydrate.

8. The conjugate of claim 7, wherein the bulking agent comprises a polysacharide.

9. The conjugate of claim 8, wherein the bulking agent comprises a dextran.

10. The conjugate of claim 1, wherein the bulking agent is covalently coupled to the photoactive compound.

11. The conjugate of claim 1, further comprising a linker between the photoactive compound and the bulking agent.

12. The conjugate of claim 11, wherein the linker is covalently bonded to the bulking agent and the photoactive compound.

13. The conjugate of claim 11, wherein the linker is structured to augment distribution of the photoactive compound within the vasculature of the patient.

14. The conjugate of claim 11, wherein the linker is structured to augment quenching of the photoactive compound when electromagnetic energy is applied to the photoactive compound.

15. The conjugate of claim 1 in a pharmaceutical composition.

16. The conjugate of claim 1, wherein the conjugate has a molecular weight of between about 60 kilodaltons and about 120 kilodaltons.

17. A method for reducing secondary damage associated with laser treatment of choroidal neovascularization, comprising a step of:
administering a conjugate to a patient having a choroidal neovascularization, the conjugate comprising a photoactive compound coupled to a bulking agent that is sized to reduce permeability of the photoactive compound through a blood vessel of the patient to reduce the amount of the photoactive compound outside of the blood vessel when electromagnetic energy from a laser is applied to the choroidal neovascularization relative to administering the photoactive compound without the bulking agent to the patient.

18. The method of claim 17, wherein the step of administering the conjugate comprises systemic administration.

19. The method of claim 17, wherein the step of administering the conjugate comprises topical administration.

20. The method of claim 19, further comprising a step of administering a permeability enhancing agent to the patient to enhance the topical administration of the conjugate to the patient relative to a similar method without administering a permeability enhancing agent to the patient.

21. The method of claim 17, wherein the step of administering the conjugate is performed at a time sufficiently before the electromagnetic energy is applied to the choroidal neovascularization to permit the conjugate to accumulate in the choroidal neovasculature.

22. A conjugate for reducing secondary damage associated with laser treatment of choroidal neovascularization, comprising:
a photoactive compound structured to absorb energy emitted from a laser; and
a leakage reducing agent coupled to the photoactive compound, the leakage reducing agent being structured to reduce leakage of the photoactive compound from a choriocapillary in an eye of a patient into the choriocapillaris of the eye of the patient after the conjugate has been administered to the patient relative to after the photoactive compound without the leakage reducing agent has been administered to the eye.

23. The conjugate of claim 22, wherein the photoactive compound comprises a porphyrin.

24. The conjugate of claim 22, wherein the photoactive compound comprises verteporfin.

25. The conjugate of claim 22, wherein the photoactive compound is selected from a group consisting of hematoporphyrins, hematoporphyrin derivatives, pheophorbides, derivatives of pheophorbides, bacteriochlorins, purpurins, merocyanines, porphycenes, and combinations thereof.

26. The conjugate of claim 22, wherein the leakage reducing agent comprises a bulking agent that is sized to sterically reduce movement of the photoactive compound from the microcapillary to the choriocapillaris of the patient.

27. The conjugate of claim 26, wherein the leakage reducing agent comprises a bulking agent selected from a group consisting of fatty acids, lipids, and carbohydrates.

28. The conjugate of claim 22, wherein the leakage reducing agent comprises a ligand that specifically binds to endothelial cells of the choriocapillaries of the patient.

29. The conjugate of claim 22, wherein the leakage reducing agent comprises a tripeptide motif of the amino acid sequence RGD.

30. The conjugate of claim 22, wherein the leakage reducing agent comprises an integrin.

31. The conjugate of claim 22, wherein the leakage reducing agent comprises Sialyl Lewis X.
